(19) United States
(12) Patent Application Publication
(10) Pub. No.: US 2026/0108650 A1
(43) Pub. Date: Apr. 23, 2026

(54) AROMA-TYPE VENTILATION TUBE

(71) Applicant: Ningbo Brightech International Trading CO., LTD, Ningbo (CN)

(72) Inventors: Jun YE, Ningbo (CN); Jinqi LU, Ningbo (CN)

(73) Assignee: Ningbo Brightech International Trading CO., LTD, Ningbo (CN)

(21) Appl. No.: 18/953,952

(22) Filed: Nov. 20, 2024

(30) Foreign Application Priority Data
Oct. 17, 2024 (CN) 202422517920.2

(51) Int. Cl. A61L 9/015

(52) U.S. Cl. CPC A61L 9/015; A61L 2209/133; A61L 2209/134

(57) ABSTRACT

The present invention discloses an aroma-type ventilation tube, which may be externally connected to an air source and blows an aroma gas, and a structure of the aroma-type ventilation tube includes a tube body, a ventilation passage arranged inside the tube body, and the like. The tube body mainly includes an aroma base, an aroma cartridge placed in the aroma base, and an aroma replaceably placed in the aroma cartridge. An aroma diffusion hole is provided on the aroma base, and an aroma ventilation hole is provided on the aroma cartridge. In this way, with rotation of the aroma cartridge in the aroma base, the aroma ventilation hole and the aroma diffusion hole are caused to be staggered or overlapped, so that the aroma gas released by the aroma cannot enter the ventilation passage or is released sequentially through the aroma ventilation hole and the aroma diffusion hole to the air passing through the ventilation passage. In addition, the aroma ventilation hole and the aroma diffusion hole can further be used for adjusting, through different overlapping areas, different amounts of the aroma gas entering the ventilation passage. Therefore, a user not only can configure various types of aromas according to actual use requirements, but also can control and adjust release of an aroma gas from the aroma to the air passing through the ventilation passage, thereby meeting use requirements in a plurality of occasions.

### CROSS-REFERENCE TO RELATED APPLICATION

This disclosure claims priority to Chinese patent application No. 202422517920.2 filed Oct. 17, 2024, the disclosure of which is incorporated herein by reference in its entirety.

### TECHNICAL FIELD

The present disclosure relates to a ventilation tube, and specifically, to an aroma-type ventilation tube.

### BACKGROUND

A ventilation tube is a tube structure externally connected to an air source for ventilation, and mainly includes a tube body and a ventilation passage that runs through the tube body and through which the air passes. Such a ventilation tube is widely used in air discharge of various types of hair dryers or new-air systems, and only performs a ventilation function. For example, the ventilation tube is used in a pet hair dryer, and may be used for blowing air to a pet, so that debris adhered to the pet are removed. However, some timid pets are easy to be scared due to being constantly blown by the wind from the ventilation tube. Alternatively, as a component of a pet hair dryer, a wind inlet hose, a motor carbon brush, or the like may carry odors of materials. These odors are mixed in the air passing through the ventilation passage and are smelled by the pet. Consequently, the pet cannot keep calm in a process of blowing. In addition, after blowing the air, the pet further needs to be additionally disinfected or sprayed with aroma to remove the smell, which is troublesome to use.

### SUMMARY

The technical problem to be resolved by the present invention is to overcome defects in the related art and provide an aroma-type ventilation tube that not only can be configured with various types of aromas according to actual use requirements, but also can control and regulate release of an aroma gas from the aroma to air passing through a ventilation passage.

The technical problem in the present invention is implemented through the following technical solutions:

An aroma-type ventilation tube, including a tube body, and a ventilation passage for air to pass through is provided inside the tube body. An aroma base, an aroma cartridge placed in the aroma base, and an aroma replaceably placed in the aroma cartridge are arranged on the tube body, where an aroma diffusion hole is provided on the aroma base, and an aroma ventilation hole is provided on the aroma cartridge. The aroma cartridge rotates in the aroma base, and drives the aroma ventilation hole of the aroma cartridge to be staggered from the aroma diffusion hole of the aroma base, to prevent an aroma gas released by the aroma from entering the ventilation passage. The aroma cartridge rotates in the aroma base, and drives the aroma ventilation hole of the aroma cartridge to overlap the aroma diffusion hole of the aroma base, so that the aroma gas released by the aroma is released sequentially through the aroma ventilation hole and the aroma diffusion hole to the air passing in the ventilation passage.

The aroma ventilation hole and the aroma diffusion hole are used for adjusting, through different overlapping areas, different amounts of aroma gas entering the ventilation passage.

A driving member is arranged at a top of the aroma cartridge, and the driving member drives the aroma cartridge to rotate in the aroma base.

An anti-detachment structure is arranged between the aroma cartridge and the aroma base, and the aroma cartridge does not detach from the aroma base while rotating relative to the aroma base by using the anti-detachment structure.

The anti-detachment structure includes a snap structure or a magnetic attraction structure. The snap structure includes a ring groove and a sliding block that are slidably assembled and respectively arranged on an outer circumferential surface of the aroma cartridge and an inner circumferential surface of the aroma base.

A seal ring forming rotational sealing of the aroma cartridge is arranged between the aroma cartridge and the aroma base.

The aroma cartridge includes a cartridge body and a cartridge cover detachably covering the cartridge body, and the aroma ventilation hole is provided at a bottom of the cartridge body.

The aroma is a solid aroma, a liquid aroma, or a gas aroma, and different types of the aroma are selected according to actual usage conditions.

The aroma base is integrally formed on a tube wall of the tube body, or a mounting hole in communication with the ventilation passage is provided on a tube wall of the tube body, and the aroma base is fitly assembled in the mounting hole.

The driving member is a lifting ring rotatably mounted on a top of the aroma cartridge.

Compared with the related art, in the present invention, an aroma base, an aroma cartridge placed in the aroma base, and an aroma replaceably placed in the aroma cartridge are mainly arranged on a tube body of a ventilation tube, and an aroma diffusion hole is provided on the aroma base, and an aroma ventilation hole is provided on the aroma cartridge. In this way, when the aroma cartridge rotates in the aroma base, and the aroma ventilation hole of the aroma cartridge is driven to be staggered from the aroma diffusion hole of the aroma base, to prevent the aroma gas released by the aroma from entering the ventilation passage. When the aroma cartridge rotates in the aroma base, and the aroma ventilation hole of the aroma cartridge is driven to be overlapped with the aroma diffusion hole of the aroma base, so that the aroma gas released by the aroma can be released sequentially, through the aroma ventilation hole and the aroma diffusion hole, to the air passing through the ventilation passage. In addition, the aroma ventilation hole and the aroma diffusion hole are used for adjusting, through different overlapping areas, different amounts of aroma gas entering the ventilation passage. Therefore, according to the foregoing structural design, a user not only can configure various types of aromas according to actual use requirements, but also can control and adjust release of an aroma gas from the aroma to the air passing through the ventilation passage, thereby meeting use requirements in a plurality of occasions. For example, the ventilation tube is used in a pet hair dryer. It is only needed to select an aroma having a calming effect, and adaptively adjust a size of an aperture of the aroma diffusion hole based on a concentration of the aroma gas received by a pet. When the pet hair dryer blows air through the ventilation tube, some timid pets are blown by the air with a calming smell and are not scared. In addition, the aroma gas can also cover odors of materials carried in a component such as an air inlet hose or a motor carbon brush, so that the pets can keep calm in a process of blowing. In addition, the aroma may be brought to the pets for aroma spraying to remove the odors. If the aroma gas contains disinfection components, the pets can be more disinfected, to avoid a trouble in subsequent use.

### BRIEF DESCRIPTION OF THE DRAWINGS

FIG. 1 is a schematic structural diagram according to the present invention;

FIG. 2 is a left view of FIG. 1;

FIG. 3 is an enlarged cross-sectional view along A-A of FIG. 1 (rotated 90°counterclockwise);

FIG. 4 is a cross-sectional view along B-B of FIG. 1;

FIG. 5 is a three-dimensional diagram of FIG. 1;

FIG. 6 is a three-dimensional exploded view of FIG. 1; and

FIG. 7 is a three-dimensional diagram of an aroma cartridge.

### DETAILED DESCRIPTION

Embodiments of the present invention are described in detail below with reference to the foregoing accompanying drawings.

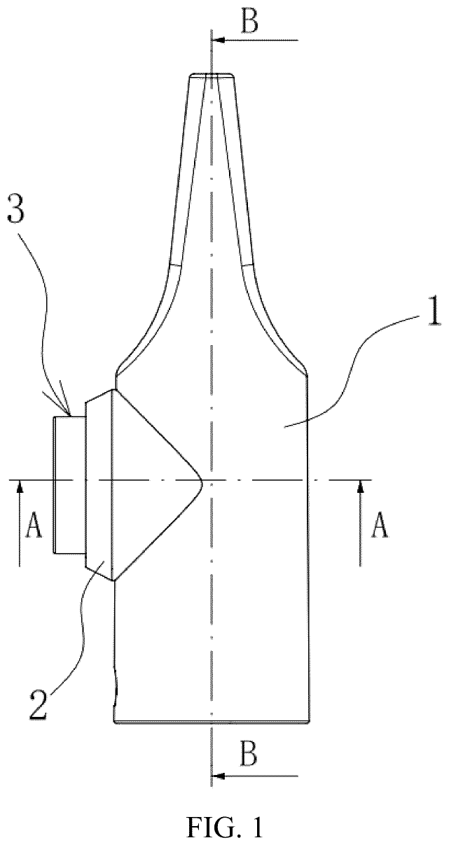

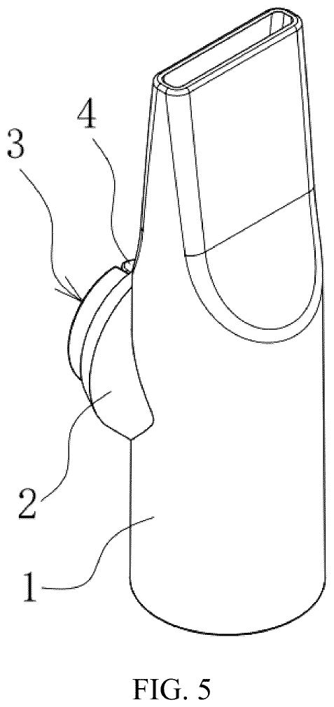

As shown in FIG. 1 to FIGS. 7, 1. tube body; 11. inlet end; 12. outlet end; 13. ventilation passage; 14. mounting hole; 2. aroma base; 21. aroma diffusion hole; 22. sliding block; 3. aroma cartridge; 31. cartridge body; 311. aroma ventilation hole; 32. cartridge cover; 33. ring groove; 4. driving member; 5. aroma; and 6. seal ring.

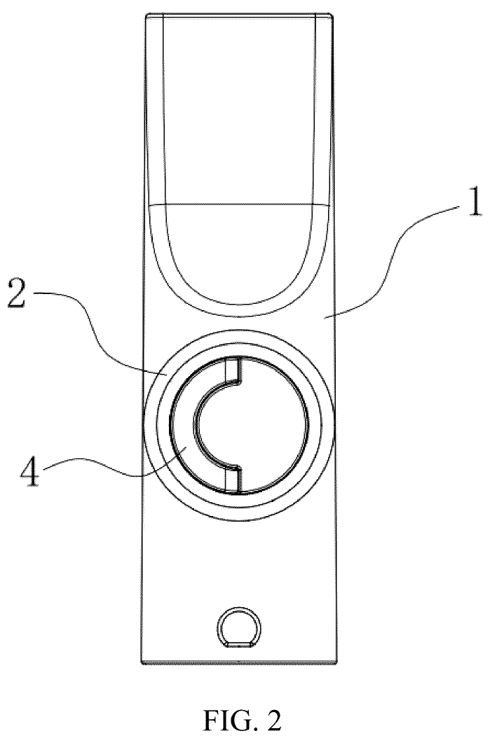

An aroma-type ventilation tube, as shown in FIG. 1 and FIG. 2, mainly relates to a ventilation tube that is externally connected to an air source and that can blow an aroma gas. The aroma-type ventilation tube may be widely used in various types of hair dryers, for example, used in a pet hair dryer, to blow air to a pet and soothe, deodorize, or disinfect the pet, or may be used in a new-air system and blow fresh air with an aroma gas, to purify the air, or may even be directly used as an independent aroma diffuser.

The aroma-type ventilation tube includes a tube body 1 in a straight tubular shape, and a ventilation passage 13 that runs through along an axis and through which air passes is arranged inside the tube body. Two ends of the tube body 1 are respectively an inlet end 11 and an outlet end 12 of the ventilation passage. In this embodiment, as shown in FIG. 1, a circular tube structure at a lower end of the view is the inlet end 11, and an air inlet hose or another air inlet attachment of an air source may be conveniently plugged, so that air can enter the ventilation passage 13 of the tube body 1. A flat tube structure at an upper end of the view is the outlet end 12, and air can be blown outward.

Certainly, the inlet end 11 and the outlet end 12 can also be designed in various manners according to actual use requirements. For example, the inlet end 11 and the outlet end 12 are designed in various structures such as a threaded pipe joint or a plug joint. Generally, the inlet end 11 takes a connection function, and the outlet end 12 may directly blow the air, or may be connected to another air outlet attachment.

An aroma base 2, an aroma cartridge 3 placed in the aroma base, and an aroma 5 replaceably placed in the aroma cartridge 3 are arranged on the tube body 1.

The aroma base 2 may be integrally formed on a tube wall of the tube body 1, or a mounting hole 14 in communication with the ventilation passage 13 may be first provided on a tube wall of the tube body 1, and then the aroma base 2 is fitly assembled in the mounting hole 14 and fixed by ultrasonic welding.

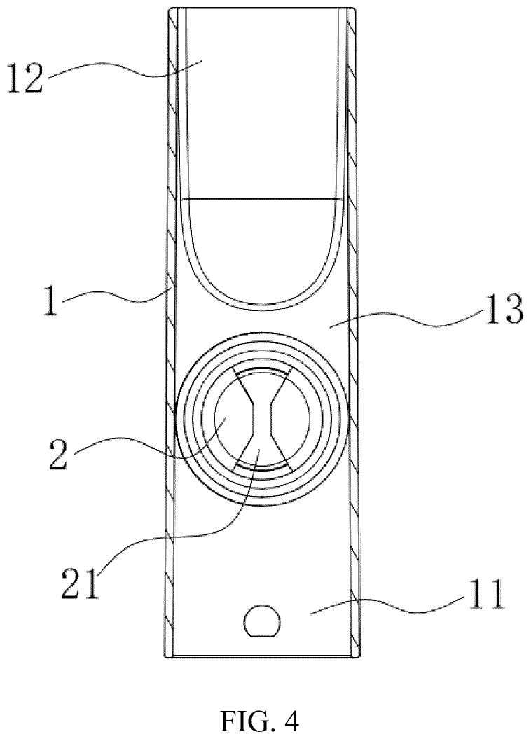

An aroma diffusion hole 21 is provided on a bottom surface of the aroma base 2. In this embodiment, as shown in FIG. 4, two sector-shaped holes that are centrosymmetrical are used as aroma diffusion holes. The two aroma diffusion holes 21 are both in communication with the ventilation passage 13, and the two aroma diffusion holes 21 are also in communication with each other.

The aroma 5 is an object that can release an aroma gas. A solid aroma may be used, for example, an aroma piece, an aroma block, or aroma particles, or a liquid aroma or a gas aroma may be used, for example, a liquid capsule-type aroma or a volatile cotton stick soaked with a liquid aroma. In other words, any aroma product that can release and volatize a smell is applicable.

Certainly, a plurality of types of smells of the aroma 5 may also be selected according to a use function in an actual situation. For example, different types of pets can respectively select aroma products with different smells or different functions.

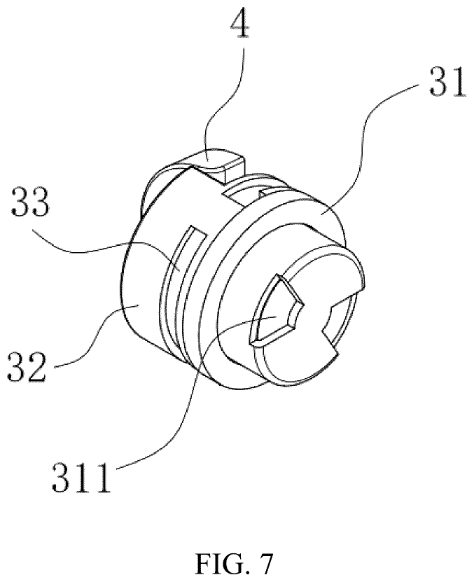

The aroma cartridge 3 includes a cartridge body 31 and a cartridge cover 32 detachably covering the cartridge body. As shown in FIG. 7, in this embodiment, the cartridge body 31 and the cartridge cover 32 mainly use a snap connection structure, which may facilitate quick connection and detachment, and also facilitate quick replacement of the aroma 5.

An aroma ventilation hole 311 is provided at a bottom of the cartridge body 31, and the aroma ventilation hole is also two sector-shaped holes that are centrosymmetrical. A shape and a size of the two aroma ventilation holes 311 are the same as that of the two aroma diffusion holes 21.

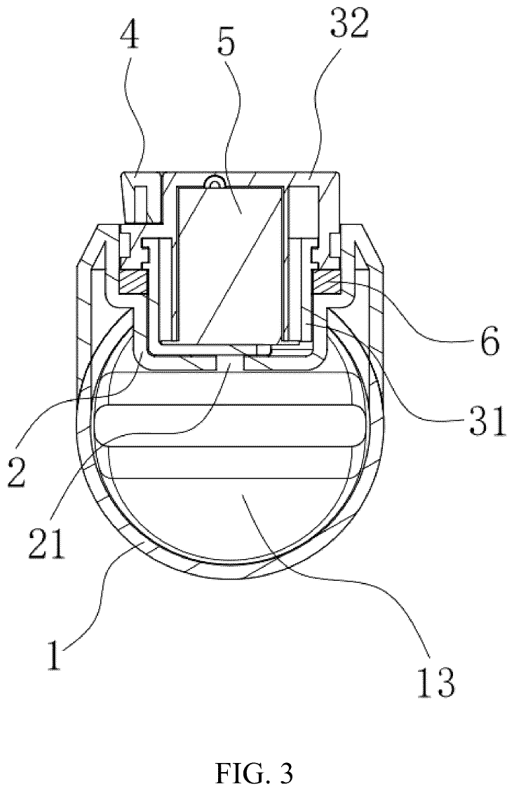

A driving member 4 is arranged at a top of the aroma cartridge 3. In this embodiment, a lifting ring is selected as shown in FIG. 3. The lifting ring not only can be used to drive the aroma cartridge 3 to rotate in the aroma base 2, but also can be conveniently taken out from the aroma base 2 when the aroma cartridge 3 is detached. In addition, the lifting ring is rotatably mounted in a hinged manner. Therefore, the lifting ring may be erected at the top of the aroma cartridge 3 when in use, to facilitate operation, and may be adhered to the top of the aroma cartridge 3 when not in use, to facilitate hiding.

When the rotation of the aroma cartridge 3 drives the two aroma ventilation holes 311 to be completely staggered from the two aroma diffusion holes 21 of the aroma base 2, the aroma gas released by the aroma 5 is prevented from entering the ventilation passage 13. This operating state is that when the aroma 5 is not detached and removed, the aroma gas is not allowed to merge into the air passing through the ventilation passage 13, and the ventilation tube can only take the function of ventilation.

When the rotation of the aroma cartridge 3 drives the two aroma ventilation holes 311 to be exactly overlapped with the two aroma diffusion holes 21 of the aroma base, the aroma 5 sequentially passes through the two aroma ventilation holes 311 and the two aroma diffusion holes 21 to release the aroma gas to the air passing through the ventilation passage 13.

In addition, with different rotation positions of the aroma cartridge 3 in the aroma base 2, overlapping areas of the two aroma ventilation holes 311 and the two aroma diffusion holes 21 are usually different. Such a rotated overlapping structure is similar to an open-close structure of a ceramics valve core in a water faucet. That is, the aroma ventilation hole 311 and the aroma diffusion hole 21 are used for adjusting, through different overlapping areas, different amounts of aroma gas entering the ventilation passage 13.

A seal ring 6 is arranged between the aroma cartridge 3 and the aroma base 2. Specifically, the seal ring is sleeved outside the aroma cartridge 3, and the seal ring 6 is located between an outer shoulder of the aroma cartridge 3 and an inner shoulder of the aroma base 2, to ensure sealing when the aroma cartridge 3 rotates. The seal ring 6 is usually a wear-resistant seal ring, which not only performs a dynamic sealing function, but also avoids affecting flexible rotation of the aroma cartridge 3.

In addition, an anti-detachment structure is further arranged between the aroma cartridge 3 and the aroma base 2, and the aroma cartridge does not detach from the aroma base 2 when being rotated relative to the aroma base by using the anti-detachment structure.

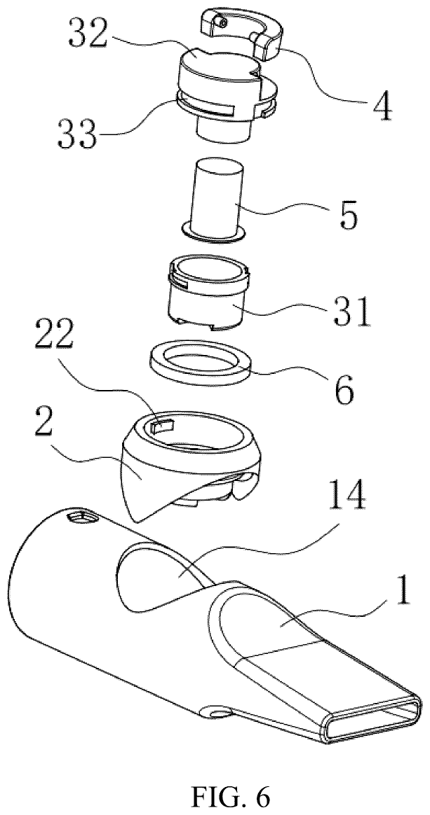

The anti-detachment structure includes a snap structure or a magnetic attraction structure. In this embodiment, the snap structure shown in FIG. 6 is selected. The snap structure mainly includes: a circular ring groove 33 provided on an outer circumferential surface of the aroma cartridge 3, and a sliding block 22 that is slidably assembled in the ring groove 33 arranged on an inner circumferential surface of the aroma base 2. In this way, when the aroma cartridge 3 is placed in the aroma base 2, and the sliding block 22 is assembled in the ring groove 33, the aroma cartridge 3 is not detached from the aroma base 2, and the aroma cartridge 3 is driven to rotate in the aroma base 2, so that the sliding block 22 can further circularly slide along the ring groove 33, thereby avoiding interference with the rotation of the aroma cartridge 3.

Certainly, the sliding block 22 may also be designed on the outer circumferential surface of the aroma cartridge 3, and the ring groove 33 is designed on the inner circumferential surface of the aroma base 2. That is, according to an actual structural situation, the ring groove 33 and the sliding block 22 that are slidably assembled and respectively arranged on the outer circumferential surface of the aroma cartridge 3 and the inner circumferential surface of the aroma base 2, and it is ensured that when the aroma cartridge 3 rotates, slidable assembly can be formed in the ring groove 33 by using the sliding block 22.

The magnetic attraction structure may be two attraction magnetic rings respectively designed in the aroma cartridge 3 and the aroma base 2, and the structure may ensure that rotation of the aroma cartridge 3 is not affected when attraction is performed. Therefore, according to the foregoing use requirements, another similar structure may also be applicable.

Several indication signs (not shown in the figure) that are circularly distributed may be arranged on a periphery of the aroma cartridge 3, and the several indication signs conveniently identify a rotation angle and a rotation position of the aroma cartridge 3 that is driven by the driving member 4, to help a user intuitively dismiss open and closed states of the aroma diffusion hole 21 and a size of an aperture after the aroma diffusion hole 21 is opened.

Therefore, according to the foregoing structural design, the user not only can configure various types of aromas 5 according to actual use requirements, but also can control and adjust the aroma 5 to release of the aroma gas 5 from the aroma to the air passing through the ventilation passage 13, thereby meeting use requirements in a plurality of occasions.

The foregoing descriptions are merely specific embodiments of the present invention, and a person skilled in the art should understand that any structural design equivalent to the embodiments shall fall within the protection scope of the present invention.

What is claimed is:

1. An aroma-type ventilation tube, comprising a tube body (1), wherein a ventilation passage (13) for air to pass through is arranged inside the tube body, wherein
an aroma base (2), an aroma cartridge (3) placed in the aroma base, and an aroma (5) replaceably placed in the aroma cartridge (3) are arranged on the tube body (1), wherein an aroma diffusion hole (21) is provided on the aroma base (2), and an aroma ventilation hole (311) is provided on the aroma cartridge (3);
the aroma cartridge (3) rotates in the aroma base (2), and drives the aroma ventilation hole (311) of the aroma cartridge (3) to be staggered from the aroma diffusion hole (21) of the aroma base (2), to prevent an aroma gas released by the aroma (5) from entering the ventilation passage (13); and
the aroma cartridge (3) rotates in the aroma base (2), and drives the aroma ventilation hole (311) of the aroma cartridge (3) to overlap the aroma diffusion hole (21) of the aroma base (2), so that the aroma gas released by the aroma (5) is released sequentially through the aroma ventilation hole (311) and the aroma diffusion hole (21) to the air passing through the ventilation passage (13).

2. The aroma-type ventilation tube according to claim 1, wherein the aroma ventilation hole (311) and the aroma diffusion hole (21) are used for adjusting, through different overlapping areas, different amounts of aroma gas entering the ventilation passage (13).

3. The aroma-type ventilation tube according to claim 2, wherein a driving member (4) is arranged at a top of the aroma cartridge (3), and the driving member drives the aroma cartridge (3) to rotate in the aroma base (2).

4. The aroma-type ventilation tube according to claim 3, wherein an anti-detachment structure is arranged between the aroma cartridge (3) and the aroma base (2), and the aroma cartridge (3) does not detach from the aroma base (2) while rotating relative to the aroma base (2) by using the anti-detachment structure.

5. The aroma-type ventilation tube according to claim 4, wherein the anti-detachment structure comprises a snap structure or a magnetic attraction structure, and the snap structure comprises a ring groove (33) and a sliding block (22) that are slidably assembled and respectively arranged on an outer circumferential surface of the aroma cartridge (3) and an inner circumferential surface of the aroma base (2).

6. The aroma-type ventilation tube according to claim 3, wherein a seal ring (6) forming rotational sealing of the aroma cartridge (3) is arranged between the aroma cartridge (3) and the aroma base (2).

7. The aroma-type ventilation tube according to claim 2, wherein the aroma cartridge (3) comprises a cartridge body (31) and a cartridge cover (32) detachably covering the cartridge body, and the aroma ventilation hole (311) is provided at a bottom of the cartridge body (31).

8. The aroma-type ventilation tube according to claim 1, wherein the aroma (5) is a solid aroma, a liquid aroma, or a gas aroma, and different types of the aroma (5) are selected according to actual usage conditions.

9. The aroma-type ventilation tube according to claim 1, wherein the aroma base (2) is integrally formed on a tube wall of the tube body (1), or a mounting hole (14) in communication with the ventilation passage (13) is provided on a tube wall of the tube body (1), and the aroma base (2) is fitly assembled in the mounting hole (14).

10. The aroma-type ventilation tube according to claim 3, wherein the driving member (4) is a lifting ring rotatably mounted at the top of the aroma cartridge (3).
